(19) United States
(12) Patent Application Publication
(10) Pub. No.: US 2022/0168317 A1
(43) Pub. Date: Jun. 2, 2022

(54) Therapeutic Thiazine Dye Compositions and Methods of Use

(71) Applicant: AB BioInnovations, Inc., Huntington, NY (US)

(72) Inventor: Alena McAllister, Huntington, NY (US)

(21) Appl. No.: 17/242,185

(22) Filed: Apr. 27, 2021

Related U.S. Application Data
(60) Provisional application No. 63/016,098, filed on Apr. 27, 2020.
(60) Provisional application No. 63/059,407, filed on Jul. 31, 2020.
(60) Provisional application No. 63/134,945, filed on Jan. 7, 2021.

(51) Int. Cl. A61K 31/5415; A61K 33/42; A61K 9/00; A61K 47/42; A61K 47/38; D06M 16/00; A61K 9/06

(52) U.S. Cl. CPC A61K 31/5415; A61K 33/42; A61K 9/0087; A61K 47/42; A61K 47/38; D10B 2401/14; A61K 9/0056; A61K 9/06; A61K 9/0014; A61K 9/0043; D10B 2401/13; D06M 16/00

(57) ABSTRACT

Therapeutic bactericidal, antiviral, anti-inflammatory, antifungal, blood clotting, and regenerative compositions comprising thiazine dye and phosphoric acid.

### CROSS-REFERENCE TO RELATED APPLICATIONS

This application claims priority to U.S. Provisional Application No. 63/016,098 filed Apr. 27, 2020, U.S. Provisional Application No. 63/059,407 filed Jul. 31, 2020, and U.S. Provisional Application No. 63/134,945 filed Jan. 7, 2021, each of which is incorporated herein by reference in its entirety.

### TECHNICAL FIELD

The present invention relates to therapeutic thiazine dye compositions and methods of use and more particularly to bactericidal, antiviral, anti-inflammatory, and regenerative compositions including methylene blue and phosphoric acid and its salts.

### BACKGROUND ART

Hospital-acquired infections, also known as nosocomial infections, are infections acquired in a hospital or other health care facility and are potentially caused by organisms that are resistant to antibiotics. Nosocomial infections represent the tenth major course of death in the United States. The annual economic impact of nosocomial infections is estimated to be in the range of 55-70 billion U.S. dollars.

No effective treatment is currently available for cases where antibiotic treatment of wounds with bacterial infections is not effective. In addition, bacteria present in an open wound are not visible to the eye. Thus, during treatment of wounds, usually a substantial amount of adjacent tissue is removed in order to minimize the likelihood of infection. Also, current surgical debridement procedures are associated with post-operative pain and swelling, do not permit an immediate reconstruction of the surgical procedure site, and require massive amounts of antibiotics for a lengthy period of time.

Osteoporotic bone and joint replacement in elderly patients is more difficult due to bone osteoporosis. Connective/supportive implants and plates do not hold in place as well due to relatively softer bone structure. In addition, an osteoporotic bone heals more slowly. In case of a fracture, it can take several months for the fracture to heal.

The present compositions ameliorate the aforementioned shortcomings of current surgical procedures in a safe, non-toxic manner.

In addition, inhalation and exhalation of air contaminated by a harmful virus or other pathogens is a common route for infection of humans. Face masks and other protective gear provide a barrier to the transmission of such pathogens.

There is an ongoing need to improve these barriers.

### SUMMARY OF THE EMBODIMENTS

In accordance with one embodiment of the invention, there is provided a therapeutic composition comprising 1.0×10⁻⁷-2.5×10⁻¹% (w/w) thiazine dye, and 1.0×10⁻⁵−2.0% (w/w) phosphoric acid. In various embodiments, the thiazine dye is methylene blue.

In accordance with another embodiment of the invention, there is provided a method of treating a subject in need thereof, the method comprising causing the administration of the therapeutic composition comprising 1.0×10⁻⁷-2.5×10⁻¹% (w/w) thiazine dye, and 1.0×10⁻⁵-2.0% (w/w) phosphoric acid. The subject may be a human or an animal.

Bioabsorbable Sponge

The present antimicrobial and anti-inflammatory composition facilitates a safe, non-toxic surgical procedure for amelioration of wound infection and inflammation, as well as enhances the ability to close a wound after a surgical procedure. In cases of bone fracture, the composition increases bone density and new bone formation as well as promotes angiogenesis at the surgical site.

In accordance with one embodiment of the invention, the antimicrobial composition comprises an aqueous solution of phosphoric acid (H₃PO₄) and an antimicrobial thiazine dye in a bioabsorbable sponge. In various embodiments, the phosphoric acid concentration in the sponge is in the range of about 0.1 percent to about 40 percent by weight of the solution depending on how the phosphoric acid-containing sponge is used.

In various embodiments, for initial cleaning of a wound, the sponge contains phosphoric acid at a concentration of about 5 to about 40 percent by weight of the aqueous solution. In various embodiments, for applications when the bioabsorbable sponge is left in a closed wound, the sponge contains phosphoric acid in an amount in the range of about 0.1 percent by weight to about 5 percent by weight based on the weight of the solution. In various embodiments, the sponge contains phosphoric acid in an amount in the range of about 0.1 percent by weight to about 2.5 percent by weight based on the weight of the solution. In various embodiments, the sponge contains phosphoric acid in an amount in the range of about 0.75 to about 2.5 percent by weight of the solution

In various embodiments, the bioabsorbable sponge contains the aqueous phosphoric acid solution in an amount in the range of about 5 to about 40 percent by weight, based on the weight of the bioabsorbable sponge.

In various embodiments, antimicrobial thiazine dye is present in the aqueous phosphoric acid solution in an amount in the range of about 0.0001 percent by weight to about 1 percent by weight. In various embodiments, antimicrobial thiazine dye is present in the aqueous phosphoric acid solution in an amount in the range of about 0.1 to about 1 percent by weight, based on the weight of the aqueous phosphoric acid solution. In various embodiments, the thiazine dye is methylene blue.

In various embodiments, the bioabsorbable sponge comprises gelatin. In various embodiments, the bioabsorbable sponge comprises collagen. In various embodiments, the aqueous solution contains a biocompatible thickening agent. In various embodiments, the biocompatible thickening agent is carboxymethyl cellulose.

Antiviral Textile

In accordance with another embodiment of the invention, an antiviral textile material that can be converted into wearing apparel such as face masks, turtle necks, scarves, bandanas, and other partial or full body coverings is described. The antiviral textile material has a hydrophilic fibrous substrate. Distributed within the fibrous substrate is a thiazine dye, such as methylene blue, and phosphoric acid. In various embodiments, the thiazine dye and phosphoric acid are present in equimolar amounts. In various embodiments, the thiazine dye is distributed in an amount in the range of 0.001 to about 1 grams per cubic centimeter of the substrate. In various embodiments, the phosphoric acid is distributed in an amount in the range of about 0.001 to about 1 grams per cubic centimeter of the substrate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1A shows the infection site of a rat treated with a 0.5% methylene blue solution, in accordance with an embodiment of the invention. FIG. 1B shows the infection site of a rat treated with a 0.5% methylene blue solution for five minutes on the entire open surgical site and after being rinsed with sterile water.

FIG. 2A shows the closed infection site of a rat treated with a 0.5% methylene blue solution, 48 hours post-op, in accordance with an embodiment of the invention. FIG. 2B shows the infection site of a negative control rat 96 hours post-infection.

FIG. 3A shows the infection site of rat treated with a 0.332031% phosphoric acid solution, in accordance with an embodiment of the invention. FIG. 3B shows the infection site of a rat treated with a 0.332031% phosphoric acid solution for five minutes on the entire open surgical site and after being rinsed with sterile water.

FIG. 4 shows the closed infection site of a rat treated with a 0.332031% phosphoric acid solution, 48 hours post-op, in accordance with an embodiment of the invention.

FIG. 5A shows the infection site of rat treated with a solution containing both 1.328125% phosphoric acid and 0.015625% methylene blue, in accordance with an embodiment of the invention. FIG. 3B shows the infection site of a rat treated with a solution containing both 1.328125% phosphoric acid and 0.015625% methylene blue for five minutes on the entire open surgical site and after being rinsed with sterile water.

FIG. 6 shows the closed infection site of a rat treated with a solution containing both 1.328125% phosphoric acid and 0.015625% methylene blue, 48 hours post-op, in accordance with an embodiment of the invention.

FIG. 7 shows the infection site of rat treated with conventional debridement of the infected site.

FIG. 8 shows the closed infection site of a rat treated with conventional debridement, 48 hours post-op, in accordance with an embodiment of the invention.

FIG. 9A shows the closed infection site of a rat treated with a 0.5% methylene blue solution, 72 hours post-op, in accordance with an embodiment of the invention. FIG. 9B shows the opened infection site of a rat treated with a 0.5% methylene blue solution, 72 hours post-op.

FIG. 10A shows the closed infection site of a rat treated with a 0.332031% phosphoric acid solution, 72 hours post-op, in accordance with an embodiment of the invention. FIG. 10B shows the opened infection site of a rat treated with a 0.332031% phosphoric acid solution, 72 hours post-op.

FIG. 11 shows the opened infection site of a rat treated with a solution containing both 1.328125% phosphoric acid and 0.015625% methylene blue, 72 hours post-op, in accordance with an embodiment of the invention.

FIG. 12 shows the opened infection site of a rat treated with conventional debridement, 72 hours post-op, in accordance with an embodiment of the invention.

FIG. 13 shows the opened infection site of a rat that received no treatment, 72 hours post-infection, in accordance with an embodiment of the invention.

FIG. 14 shows a comparison of external healing from a rat from treatment group 1, 72 hours post-op (left); treatment group 4, 72 hours post-op (center); and treatment group 5, 120 hours post-infection (right), in accordance with an embodiment of the invention.

FIG. 15 shows a perspective view of a bandage utilizing a bioabsorbable sponge in accordance with an embodiment of the invention.

FIG. 16 shows a partially exploded perspective view of a face mask having a fibrous intermediate layer with methylene blue and phosphoric acid within the fibrous intermediate layer in accordance with an embodiment of the invention.

### DETAILED DESCRIPTION OF SPECIFIC EMBODIMENTS

Definitions. As used in this description and the accompanying claims, the following terms shall have the meanings indicated, unless the context otherwise requires:

Thiazine dyes include methylene blue, azure A, azure B, toluidine blue 0, thiamine, and the like. A preferred thiazine dye is methylene blue (3,7-bis(dimethylamine)-phenothiazin-5-ium chloride.

Minimum inhibitory concentration (“MIC”) as used herein shall mean the lowest concentration of a chemical or combination of chemicals, e.g., methylene blue, phosphoric acid, and combinations thereof, which prevents visible growth of a bacteria.

As used herein with respect to Example 20, “post-op,” and the like, shall mean the time period after the second surgery (treatment) described in Example 20 herein. As used herein with respect to Example 21, “post-op,” and the like, shall mean the time period after the second surgery (treatment) described in Example 21 herein.

As used herein with respect to Example 20, “post-infection,” and the like, shall mean the time period after the first surgery described in Example 20 herein. As used herein with respect to Example 21, “post-infection,” and the like, shall mean the time period after the first surgery described in Example 21 herein. For animals that have been treated via the second surgery described in Example 20 herein, post-infection time is essentially equivalent to the post-op time plus 48 hours (because the second surgery took place 48 hours after the first surgery). For animals that have been treated via the second surgery described in Example 21 herein, post-infection time is essentially equivalent to the post-op time plus 72 hours (because the second surgery took place 72 hours after the first surgery).

Embodiments of the present invention are directed to non-toxic therapeutic compositions exhibiting surprising bactericidal, antiviral, anti-inflammatory, and regenerative properties, and which stop bleeding on the site of application.

Therapeutic compositions disclosed herein comprise one or more thiazine dyes and phosphoric acid, or salts thereof. In various embodiments, the thiazine dye is methylene blue. We have found that therapeutic compositions comprising methylene blue and phosphoric acid work synergistically to produce bactericidal, antiviral, anti-inflammatory, and regenerative effects that are not observed in compositions containing one of either methylene blue or phosphoric acid, but not both.

The combination of phosphoric acid on the methylene blue forms a bis ammonium salt (shown below) which appears to be a chemo-specific agent that has antibacterial/antimicrobial/antifungal properties. However, it also appears that it blocks (downregulates) pro-inflammatory cytokines by an unknown mechanism. The synergistic effects of these two independent mechanisms of actions may well promote the mechanisms of bone/tissue repair in a novel treatment.

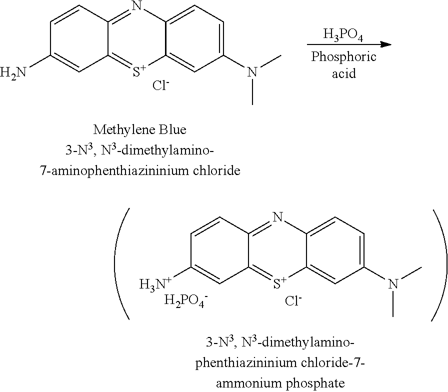

It is important to note that neither phosphoric acid nor methylene blue do this independently, but rather the combination of both is required to make the active pharmaceutical ingredient (API).

In some embodiments, therapeutic compositions comprise 1.0×10⁻⁷−2.5×10⁻¹% (w/w) thiazine dye and 1.0×10⁻⁵-2.0% (w/w) phosphoric acid. In various embodiments, the therapeutic compositions comprise 1.0×10⁻⁶-1×10⁻⁴% (w/w) thiazine dye and 1.0×10⁻⁴-1.0×10⁻²% (w/w) phosphoric acid.

In some embodiments, therapeutic compositions comprising thiazine dye and phosphoric acid can be, e.g., a mouthwash, a nasal spray, a lozenge, an inhalable therapeutic composition, a topical cream or lotion, or an acute or chronic wound treatment liquid, gel, or spray. In some embodiments, therapeutic compositions comprising thiazine dye and phosphoric acid can be used in first aid applications for open wounds as it is antibacterial, antiviral, anti-inflammatory and stops bleeding on the site of application. For example, therapeutic compositions comprising thiazine dye and phosphoric acid may be used in conjunction with wound dressings such as band aids, surgical sutures, wound gauzes, surgical sponges (e.g., bioabsorbable sponges), and the like. In some embodiments wound dressings may be pretreated with therapeutic compositions comprising thiazine dye and phosphoric acid.

In some embodiments, therapeutic compositions comprising thiazine dye and phosphoric acid can be administered prophylactically in order to reduce the risk of infection by microbes, such as bacteria and viruses. In some embodiments, prophylactic administration of compositions comprising thiazine dye and phosphoric acid protects the airways (e.g., nasal passages, sinuses, bronchial tubes, and/or lungs) and/or mouth and/or throat of a subject from infection and inflammation as the composition has been shown to have no cell toxicity. A subject can be a human or animal.

In some embodiments, therapeutic compositions comprising thiazine dye and phosphoric acid can be administered in order to treat microbial infection and/or inflammation of the airways and/or mouth and/or throat of a subject. A subject can be a human or animal.

In some embodiments, therapeutic compositions comprising thiazine dye and phosphoric acid can be administered in order to promote regeneration of damaged tissue and/or bone, increase bone density on the site of application, and induce new bone formation.

In some embodiments, therapeutic compositions comprising thiazine dye and phosphoric acid may contain one or more essential oils, dried or fresh fruit particles, suspending agents, surfactants, emollients, emulsifiers, and/or cationic polymers, e.g., as disclosed in US Publication No. 2005/0031573, which is incorporated by reference herein in its entirety.

Bioabsorbable Sponge

The present antimicrobial and anti-inflammatory bioabsorbable sponge composition can be used in conjunction with a wide variety of human and veterinary surgical interventions such as hemostasis, detection of bacterial infection, enhancement of soft tissue healing, enhancement of bone density and/or bone formation at the surgical site, enhancement of angiogenesis at the surgical site, and the like. The present bioabsorbable sponge can be also used as a foam layer in bandage or antibacterial and anti-inflammatory dressing for treatment of chronic, non-healing wounds such as chronic ulcers, diabetic lesions, and the like, as first aid in remote or battlefield regions, as well as for post extraction wound care in dentistry, for bacterial detection in wounds, and the like.

The term “bioabsorbable sponge” as used herein means a water-insoluble, pliable, absorbent biocompatible material that, when implanted in a human or other mammalian body, is absorbed by the body. In various embodiments, the bioabsorbable sponge is non-immunogenic.

Illustrative bioabsorbable sponges can have a gelatin matrix, a collagen matrix, a poly-L-lactic acid matrix, and the like. Particularly well suited for present purposes is a bioabsorbable sponge commercially available under the designation GEL-FOAM® from Pfizer, Inc. as a water-insoluble, porous, pliable product prepared from purified pork skin gelatin USP.

The bioabsorbable sponge is impregnated with the aqueous phosphoric acid solution. In some embodiments, the aqueous phosphoric acid solution constitutes at least 5 percent by weight of the bioabsorbable sponge. In some embodiments, the aqueous phosphoric acid solution constitutes about 5 to about 40 percent by weight of the sponge. In some embodiments, the aqueous phosphoric acid solution constitutes about 10 to about 30 percent by weight of the sponge.

The aqueous phosphoric acid solution also contains an antimicrobial thiazine dye, such as methylene blue, azure A, azure B, toluidine blue 0, thianine, and the like. A preferred thiazine dye is methylene blue (3,7-bis(dimethylamine)-phenothiazin-5-ium chloride).

In various embodiments, the antimicrobial thiazine dye is present in the aqueous phosphoric acid solution in an amount in the range of about 0.0001 to about 1 weight percent, based on the weight of the solution. In various embodiments, the antimicrobial thiazine dye is present in the aqueous phosphoric acid solution in an amount in the range of about 0.1 to about 1 weight percent, based on the weight of the solution. In various embodiments, the antimicrobial thiazine dye is present in the aqueous phosphoric acid solution in an amount in the range of about 0.01 to about 0.1 weight percent, based on the weight of the solution. The thiazine dye is released from the bioabsorbable sponge to the surrounding tissue as the sponge is liquefied and absorbed by the surrounding tissue. The thiazine dye also serves as a color indicator because if bacteria are present in the wound these bacteria will be stained dark or black, and can be surgically removed before a surgical procedure is performed and the wound is closed.

For wound pretreatment, a biocompatible thickener can also be added to the aqueous acid solution and the phosphoric acid content can be in the range of about 20 to about 40 weight percent, based on the weight of the solution. In various embodiments, a thickened aqueous phosphoric acid solution has a viscosity of about 100 to about 2000 contipoises (cp). In various embodiments, the thickener is sodium carboxymethyl cellulose having an average degree of substitution of about 21% to about 33%.

Antiviral Textile

As used herein, the term “apparel” means textile wearing apparel positionable over the mouth and nasal passages of a human, e.g., a mask, a turtle neck, a scarf, a bandana, and the like, as well as a full or partial body covering.

As used herein, the term “textile” means a fibrous artifact made by weaving, felting or crocheting natural fibers, synthetic fibers, and mixtures thereof.

A textile material suitable as a substrate for the thiazine dye and phosphoric acid is hydrophilic and fibrous, and sufficiently air permeable or porous to permit a human to breathe therethrough. The textile material can be woven material or a non-woven material. Preferred are non-woven materials made of cotton, cellulose, polypropylene, and the like, having a density of about 20 to 25 grams per square meter, and filtering particles having a size of 1 micron and larger. In various embodiments, the textile material filters particles having a size of 0.3 microns and larger.

In various embodiments, the amount of thiazine dye distributed within the fibrous substrate of the textile material is in the range of about 0.001 to about 1 grams per cubic centimeter (cm³) of the substrate. In various embodiments, the amount of thiazine dye distributed within the fibrous substrate of the textile material is in the range of about 0.1 to about 0.5 grams per cubic centimeter of the substrate.

In various embodiments, the amount of phosphoric acid distributed within the fibrous substrate of the textile material is in the range of 0.001 to about 1 grams per cubic centimeter of the substrate.

In various embodiments, an equimolar amount of thiazine dye and phosphoric acid is present in the fibrous substrate. The thiazine dye may be methylene blue.

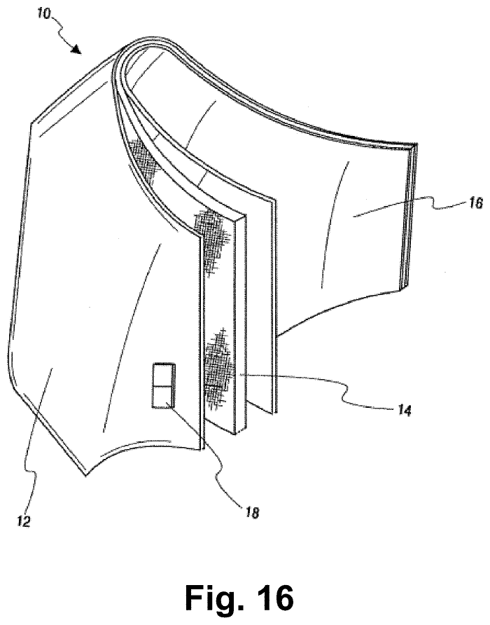

Referring to FIG. 16, face mask 10 has an outer layer 12 made of water repellant cotton, an intermediate layer 14 made of non-woven cotton, an inner layer 16 made of silk. Intermediate layer 14 is hydrophilic and provides a fibrous substrate into which methylene blue and phosphoric acid are distributed. Aperture 18 in outer layer 12 is provided for attachment of an appropriate securing strap.

During use on a facial covering, the antiviral textile material is moistened by exhaled moisture. In some instances, especially when ambient humidity is relatively low, it is desirable to moisten the facial covering. This can be achieved by applying a mist or spray of water to the facial covering prior to use. Alternatively, an antiviral solution containing a thiazine dye and phosphoric acid can be applied to the facial covering prior to use or at intervals during use, depending on ambient conditions. In various embodiments, the antiviral solution suitable for this purpose contains about 0.1 to about 5 weight percent of phosphoric acid based on the weight of the antiviral solution. In various embodiments, the antiviral solution contains about 0.75 to about 2.5 weight percent based on the weight of the antiviral solution. In various embodiments, the antiviral thiazine dye is present in the aqueous solution in an amount in the range of about 0.01 to about 1 weight percent based on the weight of the antiviral solution.

### Example 1: Therapeutic Formulation #1

A therapeutic formulation in accordance with embodiments of the invention is prepared as follows.

Methylene Blue Solution A is prepared by diluting 85 μl of a 1% (w/v) methylene blue solution in water to a total volume of 1000 ml, resulting in a Methylene Blue Solution A concentration of 8.5×10⁻⁵ (w/v).

Phosphoric Acid Solution A is prepared by diluting 1 g of 85% (w/w) phosphoric acid in water to a total volume of 850 ml, resulting in a Phosphoric Acid Solution A concentration of 1.0×10⁻¹% (w/v).

Therapeutic Formulation #1 is created by combining 86.6 g of Methylene Blue Solution A, 0.2 g of Phosphoric Acid Solution A, and 13.2 g of water. The resulting composition comprises methylene blue at a final concentration of approximately 7.4×10⁻⁵% (w/w) and phosphoric acid at a final concentration of approximately 2.0×10⁻³% (w/w). We have found that this composition has bactericidal, antiviral, regenerative, and anti-inflammatory properties in human and animal subjects.

### Example 2: Therapeutic Formulation #2

A therapeutic formulation in accordance with embodiments of the invention is prepared as follows.

Methylene Blue Solution B is prepared by diluting 3.0 μl of a 1% (w/v) methylene blue solution in water to a total volume of 1000 ml, resulting in a Methylene Blue Solution B concentration of 3.0×10⁻⁶% (w/v).

Phosphoric Acid Solution A is prepared by diluting 1 g of 85% (w/w) phosphoric acid in water to a total volume of 850 ml, resulting in a Phosphoric Acid Solution A concentration of 1.0×10⁻¹ (w/v).

Therapeutic Formulation #2 is created by combining 86.6 g of Methylene Blue Solution B, 0.2 g of Phosphoric Acid Solution A, and 13.2 g of water. The resulting composition comprises methylene blue at a final concentration of approximately 2.6×10⁻⁶% (w/w) and phosphoric acid at a final concentration of approximately 2.0×10⁻³% (w/w). This composition has the same constituents as in Example 1, but the concentration of methylene blue is much lower than in Example 1. Even when the concentration of methylene blue has the concentrations of this Example 2, we have found that this composition has bactericidal, antiviral, regenerative, and anti-inflammatory properties in human and animal subjects.

### Example 3: Mouthwash Formulation #1

An oral hygiene mouthwash in accordance with embodiments of the invention was prepared as follows.

Methylene Blue Solution A was prepared by diluting 85 μl of a 1% (w/v) methylene blue solution in water to a total volume of 1000 ml, resulting in a Methylene Blue Solution A concentration of 8.5×10⁻⁵% (w/v).

Phosphoric Acid Solution A was prepared by diluting 1 g of 85% (w/w) phosphoric acid in water to a total volume of 850 ml, resulting in a Phosphoric Acid Solution A concentration of 1.0×10⁻¹ (w/v).

Mouthwash #1 was created by first combining the materials of Phase A, shown in Table 1, below. The Phase A mixture was then combined with the material of Phase B, shown in Table 1, below.

TABLE 1
Mouthwash #1 Formulation

| | |
|---|---|
| Grams | Material |

PHASE A

| | |
|---|---|
| 86.6 | Methylene Blue Solution A |
| 0.2 | Phosphoric Acid Solution A |
| 5.0 | Aloe Vera Gel |
| 0.5 | Propandiol 1,3 |
| 0.25 | Myrrh Gum (in Glycerin) |
| 0.25 | Sage Leaf (in Glycerin) |
| 0.25 | Organic Peppermint Extract |
| 0.25 | Rosemary Extract |
| 5.0 | Glycerin |

PHASE B

| | |
|---|---|
| 1.7 | Benzoic Acid |
| 100 g Total | |

The resulting mouthwash comprises methylene blue at a final concentration of approximately 7.4×10⁻⁵% (w/w) and phosphoric acid at a final concentration of approximately 2.0×10⁻³% (w/w).

We have found that this composition has bactericidal, antiviral, regenerative, and anti-inflammatory properties in human and animal subjects.

### Example 4: Mouthwash Formulation #2

An oral hygiene mouthwash in accordance with embodiments of the invention was prepared as follows.

Methylene Blue Solution B was prepared by diluting 3.0 μl of a 1% (w/v) methylene blue solution in water to a total volume of 1000 ml, resulting in a Methylene Blue Solution B concentration of 3.0×10⁻⁶% (w/v).

Phosphoric Acid Solution A was prepared by diluting 1 g of 85% (w/w) phosphoric acid in water to a total volume of 850 ml, resulting in a Phosphoric Acid Solution A concentration of 1.0×10⁻¹ (w/v).

Mouthwash #2 was created by first combining the materials of Phase A shown in Table 2, below. The Phase A mixture was then combined with the material of Phase B, shown in Table 2, below.

TABLE 2
Mouthwash #2 Formulation

| | |
|---|---|
| Grams | Material |

PHASE A

| | |
|---|---|
| 86.6 | Methylene Blue Solution B |
| 0.2 | Phosphoric Acid Solution A |
| 5.0 | Aloe Vera Gel |
| 0.5 | Propandiol 1,3 |
| 0.25 | Myrrh Gum (in Glycerin) |
| 0.25 | Sage Leaf (in Glycerin) |
| 0.25 | Organic Peppermint Extract |
| 0.25 | Rosemary Extract |
| 5.0 | Glycerin |

PHASE B

| | |
|---|---|
| 1.7 | Benzoic Acid |
| 100 g Total | |

The resulting mouthwash comprises methylene blue at a final concentration of approximately 2.6×10⁻⁶% (w/w) and phosphoric acid at a final concentration of approximately 2.0×10⁻³% (w/w).

Even when the concentration of methylene blue has the concentrations of this Example 4, we have found that this composition has bactericidal, antiviral, regenerative, and anti-inflammatory properties in human and animal subjects.

### Example 5: Nasal Spray Formulation

A therapeutic nasal spray in accordance with embodiments of the invention was prepared as follows.

Methylene Blue Solution A was prepared by diluting 85 μl of a 1% (w/v) methylene blue solution in water to a total volume of 1000 ml, resulting in a Methylene Blue Solution A concentration of 8.5×10⁻⁵% (w/v).

Phosphoric Acid Solution A was prepared by diluting 1 g of 85% (w/w) phosphoric acid in water to a total volume of 850 ml, resulting in a Phosphoric Acid Solution A concentration of 1.0×10⁻¹ (w/v).

The therapeutic nasal spray was created by first combining the materials of Phase A, shown in Table 3, below. Next, the materials of Phase B, shown below in Table 3, were combined. Finally, the Phase A mixture was combined with the Phase B mixture.

TABLE 3
Nasal Spray Formulation

| | |
|---|---|
| Grams | Material |

PHASE A

| | |
|---|---|
| 4.935 | Methylene Blue Solution A |
| 0.3 | Phosphoric Acid Solution A |
| 90.0 | Water |
| 0.4 | Sea Salt |
| 0.1 | Allantoin |
| 0.25 | Aloe Vera Gel |
| 2.0 | Glycerin |
| 0.1 | Xanthan Gum |
| 0.2 | Panthenol |
| 0.5 | Propandiol 1,3 |
| 0.025 | Ginkgo Biloba |
| 0.025 | Hibiscus |
| 0.025 | Rice Bran Protein |
| 0.025 | Birchwood Extract |
| 0.025 | Green Tea Extract |
| 0.025 | Calendula Extract |
| 0.025 | Colloidal Oat |

PHASE B

| | |
|---|---|
| 0.01 | Grapefruit Seed Oil |
| 0.01 | Eucalyptus Oil |
| 0.01 | Peppermint Oil |
| 0.01 | Tea Tree Oil |
| 1.0 | Grapefruit Seed Extract |
| 100 g Total | |

The resulting nasal spray comprises methylene blue at a final concentration of approximately 4.2×10⁻⁶% (w/w) and phosphoric acid at a final concentration of approximately 3.0×10⁻³% (w/w).

We have found that this composition has bactericidal, antiviral, regenerative, and anti-inflammatory properties in human and animal subjects.

### Example 6: Use of Therapeutic Formulation #1 to Reduce the Number of Microorganisms in a Patient's Mouth During Oral Procedures

To reduce the number of microorganisms, including viruses, in a patient's mouth during oral procedures, the patient is administered the therapeutic formulation of Example 1 just prior to an oral procedure, thereby decreasing the risk of infection for the patient and decreasing the risk of cross infection to health care staff. It reduces periodontal infections and gingival swelling (gingivitis) induced by oral pathogens.

Just prior to an oral procedure, the patient vigorously swishes 10 ml of the therapeutic formulation of Example 1 around their mouth for 1 minute and then spits it out.

### Example 7: Use of Therapeutic Formulation #2 to Reduce the Number of Microorganisms in a Patient's Mouth During Oral Procedures

To reduce the number of microorganisms, including viruses, in a patient's mouth during oral procedures, the patient is administered the therapeutic formulation of Example 2 just prior to an oral procedure, thereby decreasing the risk of infection for the patient and decreasing the risk of cross infection to health care staff. It should be used immediately after the treatment to avoid post-treatment bleeding, inflammation, swelling and pain, and to reduce the time of the healing.

Just prior to an oral procedure, the patient vigorously swishes 10 ml of the therapeutic formulation of Example 2 around their mouth for 1 minute and then spits it out.

### Example 8: Use of Mouthwash #1 to Reduce the Number of Microorganisms in a Patient's Mouth During Oral Procedures

To reduce the number of microorganisms, including viruses, in a patient's mouth during oral procedures, the patient is administered the mouthwash of Example 3 just prior to an oral procedure, thereby decreasing the risk of infection for the patient and decreasing the risk of cross infection to health care staff.

Just prior to an oral procedure, the patient vigorously swishes 10 ml of the mouthwash of Example 3 around their mouth for 1 minute and then spits it out.

### Example 9: Use of Mouthwash #2 to Reduce the Number of Microorganisms in a Patient's Mouth During Oral Procedures

To reduce the number of microorganisms, including viruses, in a patient's mouth during oral procedures, the patient is administered the mouthwash of Example 4 just prior to an oral procedure, thereby decreasing the risk of infection for the patient and decreasing the risk of cross infection to health care staff.

Just prior to an oral procedure, the patient vigorously swishes 10 ml of the mouthwash of Example 4 around their mouth for 1 minute and then spits it out.

### Example 10: Use of Nasal Spray to Prophylactically Reduce the Risk of Infection

In order to prophylactically reduce the risk of infection, for example, during a pandemic, a human subject administers the nasal spray formulation of Example 5 into each nostril.

To reduce the risk of bacterial and viral infection, the human subject administers 0.1-1.0 ml of the nasal spray formulation of Example 5 into each nostril at least once per day. The nasal spray is administered using nasal spray devices well-known in the art. The nasal spray formulation coats the lining of the nasal passages, sinuses, and other proximal airway tissues, exerting bactericidal and antiviral effects on these membranes and tissues. The formulation reduces inflammation-induced secretion and opens air passages.

### Example 11: Use of Nasal Spray to Prophylactically Reduce the Risk of Infection

In order to prophylactically reduce the risk of infection, for example, during a pandemic, a human subject administers the nasal spray formulation of Example 5 into each nostril.

To reduce the risk of bacterial and/or viral infection, the human subject administers 0.1-1.0 ml of the nasal spray formulation of Example 5 into each nostril at least once per day as needed. The nasal spray is administered using nasal spray devices well-known in the art. The nasal spray formulation coats the lining of the nasal passages, sinuses, and other proximal airway tissues, exerting bactericidal and antiviral effects on these membranes and tissues.

### Example 12: Use of Nasal Spray to Treat Infection

In order to treat a human subject having a bacterial and/or viral infection of the nasal passages or upper airways, the nasal spray formulation of Example 5 is administered into each nostril.

To treat a human subject having a bacterial and/or viral infection, the human subject administers 0.1-1.0 ml of the nasal spray formulation of Example 5 into each nostril at least once per day post-infection for up to four weeks. The nasal spray is administered using nasal spray devices well-known in the art. The nasal spray formulation coats the lining of the nasal passages, sinuses, and other proximal airway tissues, exerting bactericidal, antiviral, anti-inflammatory, and regenerative effects on these membranes and tissues.

### Example 13: Use of Inhaled Formulation to Prophylactically Reduce the Risk of Infection

In order to prophylactically reduce the risk of infection, for example, during a pandemic, a human subject inhales the nasal spray formulation of Example 5.

To reduce the risk of bacterial and/or viral infection, the human subject inhales 0.1-1.0 ml of the nasal spray formulation of Example 5 at least once per day as needed using inhaler or nebulizer devices well-known in the art. The nasal spray formulation coats the lining of the bronchial tubes and lungs, exerting bactericidal and antiviral effects on these membranes and tissues.

### Example 14: Use of Inhaled Formulation Spray to Treat Infection

In order to treat a human subject having a bacterial and/or viral infection of the bronchial tubes or lungs, the nasal spray formulation of Example 5 is inhaled by the human subject.

To treat a human subject having a bacterial and/or viral infection, the human subject inhales 0.1-1.0 ml of the nasal spray formulation of Example 5 at least once per day post-infection for up to four weeks using inhaler or nebulizer devices well-known in the art. The nasal spray formulation coats the lining of the bronchial tubes and lungs, exerting bactericidal, antiviral, anti-inflammatory, and regenerative effects on these membranes and tissues.

### Example 15: Use of Therapeutic Skin Cream to Cleanse and Regenerate Skin Tissue

In order to cleanse (sterilize) and regenerate the skin of a human or animal subject, a topical cream comprising methylene blue at a final concentration of approximately 7.4×10⁻⁵ (w/w) and phosphoric acid at a final concentration of approximately 2.0×10⁻³% (w/w) is applied to the skin at the area of interest. The topical cream is similar to the formulation of Example 1, but comprises lipids and other topical cream components well-known in the art. The topical cream is applied to the area of interest at least once per day as needed.

Alternatively, the topical cream comprises methylene blue at a final concentration of approximately 2.6×10⁻⁶% (w/w) and phosphoric acid at a final concentration of approximately 2.0×10⁻³%. The topical cream is similar to the formulation of Example 2, but comprises lipids and other topical cream components well-known in the art. The topical cream is applied to the area of interest at least once per day as needed.

### Example 16: Use of Therapeutic Skin Cream to Treat Inflammation and/or Infection of the Skin

In order to treat inflammation and/or infection and/or open acute and non-healing wounds (e.g., diabetics and bed sores) of the skin of a human or animal subject, a topical cream comprising methylene blue at a final concentration of approximately 7.4×10⁻⁵% (w/w) and phosphoric acid at a final concentration of approximately 2.0×10⁻³% (w/w) is applied to the skin at the area of interest. The topical cream is similar to the formulation of Example 1, but comprises lipids and other cream components well-known in the art. The topical cream is applied to the area of interest at least once per day as needed.

Alternatively, the topical cream comprises methylene blue at a final concentration of approximately 2.6×10⁻⁶% (w/w) and phosphoric acid at a final concentration of approximately 2.0×10⁻³%. The topical cream is similar to the formulation of Example 2, but comprises lipids and other topical cream components well-known in the art. The topical cream is applied to the area of interest at least once per day as needed.

### Example 17: Use of Therapeutic Lozenge to Prophylactically Reduce the Risk of Infection

In order to prophylactically reduce the risk of infection, for example, during a pandemic, a human subject dissolves one lozenge slowly in the mouth, repeating as needed. The lozenge comprises methylene blue at a final concentration of approximately 7.4×10⁻⁵% (w/w) and phosphoric acid at a final concentration of approximately 2.0×10⁻³% (w/w). The lozenge may further comprise sugars, starches, and flavorings.

Alternatively, the lozenge comprises methylene blue at a final concentration of approximately 2.6×10⁻⁶% (w/w) and phosphoric acid at a final concentration of approximately 2.0×10⁻³% (w/w).

The dissolved lozenge coats the lining of the mouth and throat, exerting bactericidal and antiviral effects on these membranes and tissues.

### Example 18: Use of Therapeutic Lozenge to Prophylactically Reduce the Risk of Infection

In order to treat a human subject having a bacterial and/or viral infection of the mouth or throat, a human subject dissolves one lozenge slowly in the mouth, repeating at least once a day as needed. The lozenge comprises methylene blue at a final concentration of approximately 7.4×10⁻⁵% (w/w) and phosphoric acid at a final concentration of approximately 2.0×10⁻³% (w/w). Methylene blue also provides a numbing effect. The lozenge may further comprise sugars, starches, and flavorings.

Alternatively, the lozenge comprises methylene blue at a final concentration of approximately 2.6×10⁻⁶% (w/w) and phosphoric acid at a final concentration of approximately 2.0×10⁻³% (w/w).

The dissolved lozenge coats the lining of the mouth and throat, exerting bactericidal, antiviral, anti-inflammatory, and regenerative effects on these membranes and tissues.

### Example 19: Minimum Inhibitory Concentrations (MIC) of Phosphoric Acid and Methylene Blue on Select Bacterial Species

Starting with an 85% phosphoric acid solution, a 2-fold serial dilution series was generated consisting of 24 members having a phosphoric acid concentration ranging from 85% to 1.0132789612×10⁻⁵%. Similarly, starting with a 1% methylene blue solution, a 2-fold serial dilution series was generated consisting of 24 members having a methylene blue concentration ranging from 1% to 1.1920929×10⁻⁷%. Each dilution series was prepared using Cation-Adjusted Muller Hinton broth (CAMHB).

For each dilution series, 0.1 ml of each dilution, in order from most concentrated to least concentrated, was placed into a separate well of a 96-well tissue-culture plate. Select bacterial species were diluted in CAMHB so that 0.1 ml contained approximately 1×10⁶ bacteria per ml. Next, 0.1 ml of diluted bacterial suspension was added to each well containing the phosphoric acid or methylene blue. The tissue-culture plates were incubated at 37° C. for 24 hours.

After 24 hours, each well was visually assessed using a light microscope for bacterial growth. For each compound, the dilution having the lowest concentration capable of inhibiting bacterial growth was defined as the MIC. These results are presented in Table 4, below, the percentages being the final concentration of the compound in the 0.2 ml of total liquid in each well.

TABLE 4
MIC of Methylene Blue and Phosphoric Acid

| Bacteria Species | Methylene Blue MIC | Phosphoric Acid MIC |
|---|---|---|
| Pseudomonas aeruginosa | 0.5% | 0.3320312% |
| ATCC 27853 | | |
| Klebsiella pneumoniae | 0.25% | 0.3320312% |
| ATCC 13883 | | |
| Staphylococcus aureus | 0.0039062% | 0.3320312% |
| ATCC BAA-44 | | |

In a separate experiment, equal volumes of 85% phosphoric acid and 1% methylene blue were mixed together (1:1). Starting with this mixture, a 2-fold serial dilution series was generated using CAMHB consisting of 24 members having a phosphoric acid/methylene blue concentration ranging from 42.5%/0.5% to 5.066394806×10⁻⁶%/5.9604645×10⁻⁸%, respectively.

0.1 ml of each dilution, in order from most concentrated to least concentrated, was placed into an individual well of a 96-well tissue-culture plate. Select bacterial species were diluted in CAMHB so that 0.1 ml contained approximately 1×10⁶ bacteria per ml. Next, 0.1 ml of diluted bacterial suspension was added to each well containing the phosphoric acid and methylene blue. The tissue-culture plates were incubated at 37° C. for 24 hours.

After 24 hours, each well was visually assessed using a light microscope for bacterial growth. The dilution with the lowest concentrations capable of inhibiting bacterial growth was defined as the MIC. These results are presented in Table 5, below, the percentages being the final concentration of each compound in the 0.2 ml of total liquid in each well.

TABLE 5
MIC of a Phosphoric Acid/Methylene Blue Mixture

| | Phosphoric Acid/Methylene |
|---|---|
| Bacteria Species | Blue Mixture MIC |
| Pseudomonas aeruginosa | 0.6640625%/0.0078125% |
| ATCC 27853 | |
| Staphylococcus aureus | 0.3320312%/0.0039062% |
| ATCC BAA-44 | |

The protocol, above, was a modification of the microbroth dilution assay of the Clinical and Laboratory Standard Institute (CLSI), which is incorporated by reference herein in its entirety. CLSI. Approved Standard M7-A6. 6. Clinical and Laboratory Standards Institute; Wayne, Pa.: 2006. Methods for Dilution Antimicrobial Susceptibility Tests for Bacteria that Grow Aerobically.

### Example 20: In Vivo Testing of the Bactericidal/Antibacterial Properties of Methylene Blue, Phosphoric Acid, and a Mixture of Phosphoric Acid and Methylene Blue in Rats

To assess the ability of a mixture of phosphoric acid and methylene blue to prevent infection and promote healing, in vivo experiments were performed on rats. For these experiments, two surgeries were performed on each rat tested.

In the first surgery, the rear right thigh of each animal was infected by intramuscular injection of 0.2 ml of Pseudomonas aeruginosa ATCC 27853 at a concentration of 3.8×10⁵ per ml. Animals were left to develop the infection for 48 hours. All animals developed inflammation (swelling, change of the skin color (bluish, purple)) at the injection site and were limping on the infected leg.

In the second surgery, under sedation, the infected area was surgically opened (except for treatment group 5) and the treatments shown in Table 6 were applied onto the entire area of the open wound for the 5 minutes. Treatment solutions were prepared using sterile water. After five minutes, the entire wound area was rinsed with sterile water.

TABLE 6
Treatment Groups

| Treatment Group | Treatment |
|---|---|
| 1 | 0.5% methylene blue solution (pH pH 7.8) |
| 2 | 0.332031% phosphoric acid (pH 1.23) |
| 3 | 1.328125% phosphoric acid + 0.015625% |
| | methylene blue solution (pH 0.99) |
| 4 (Positive Control) | Conventional debridement with sterile |
| | water rinse |
| 5 (Negative Control) | Not treated |

Treatment Group 1

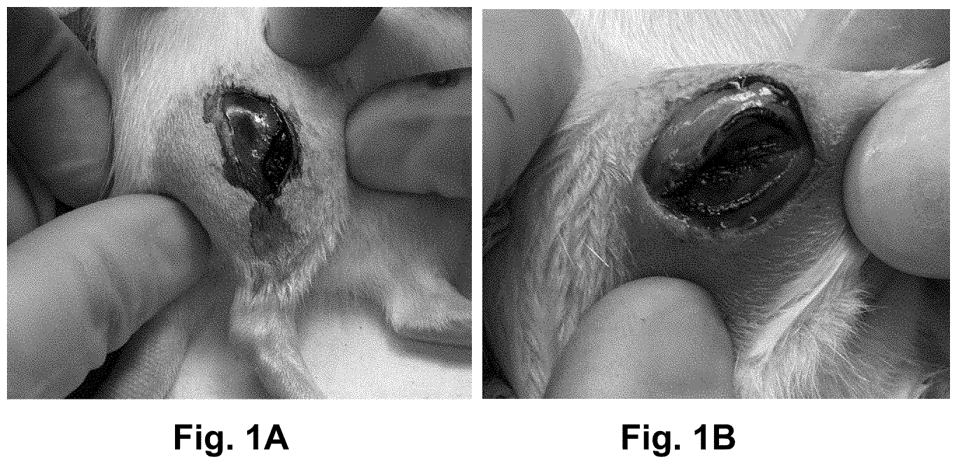

FIG. 1A shows the surgical site after application of the 0.5% methylene blue solution to the entire open surgical area after 5 minutes. Application of the 0.5% methylene blue solution stopped bleeding on the site.

FIG. 1B shows the surgical site after the after the 5 minute application of 0.5% methylene blue and after being rinsed with sterile water. The infected tissue area is clearly stained blue. Stained tissue was surgically removed and the site was rinsed one more time with sterile water until completely stain-free. The wound was closed with internal resorbable stiches and skin staples. Animals received 1 ml saline solution subcutaneously and 0.03 mg/kg of buprenorphine for pain. Buprenorphine was then administered at 0.02 mg/kg every 12 hours for the next 48 hours. Anti-inflammatory pain killers were avoided.

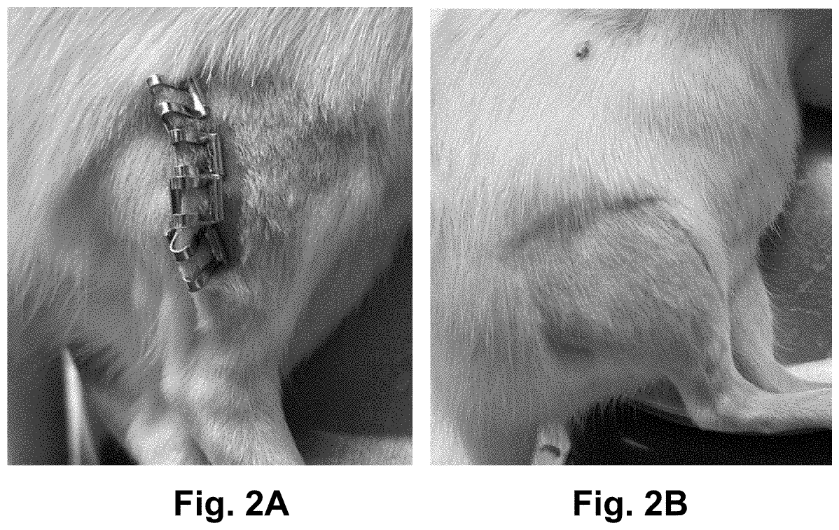

Treatment group 1, 48 hours post-op. As shown in FIG. 2A, 48 hours after treatment with the 0.5% methylene blue solution, the animals developed a higher degree of inflammation in general compared to the negative control (treatment group 5, shown in FIG. 2B). Animals treated with 0.5% methylene blue solution showed fur loss, fluctuation of the infected site, and increased limping. Animals treated with 0.5% methylene blue were the most lethargic compared to all other treatment groups (general signs of illness). Furthermore, the limping on the infected leg increased.

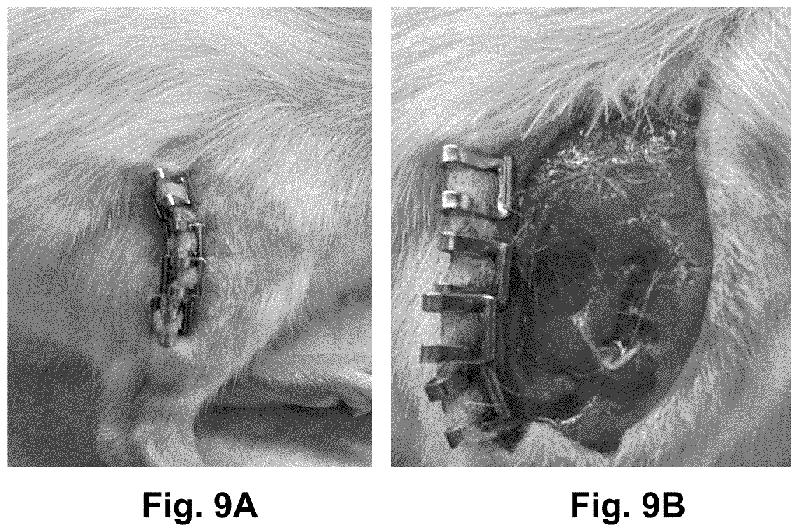

Treatment group 1, 72 hours post-op. FIG. 9A shows the unopened surgical site and FIG. 9B shows the opened surgical site of an animal 72 hours after treatment with the 0.5% methylene blue solution. Swelling was observed at the surgical area and at adjacent tissues. The surgical area showed inflamed tissues.

Treatment Group 2

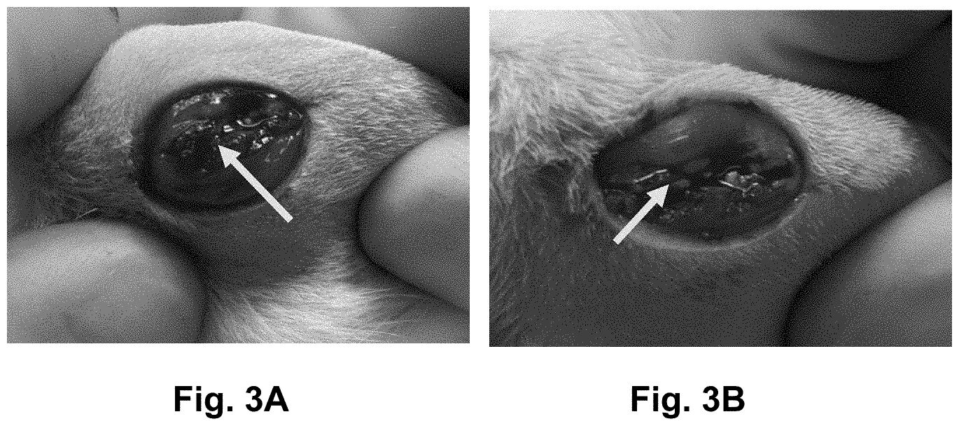

FIG. 3A shows the surgical site after application of the 0.332031% phosphoric acid solution to the entire open surgical site after 5 minutes. Application of the 0.332031% phosphoric acid solution stopped bleeding on the site.

FIG. 3B shows the surgical site after the after the 5 minute application of 0.332031% phosphoric acid and after being rinsed with sterile water. No bleeding was observed. As exemplified by the tissue being pointed to by the yellow arrow in FIG. 3A and FIG. 3B, the infected tissue area appears “curled” and is distinctively different from the healthy tissues surrounding it. Healthy tissues did not appear to be affected by the 0.332031% phosphoric acid solution. The observed curled tissues were surgically removed. The site was rinsed again with sterile water the wound was closed with internal resorbable stiches and skin staples. Animals received 1 ml saline solution subcutaneously and 0.03 mg/kg of buprenorphine for pain. Buprenorphine was then administered at 0.02 mg/kg every 12 hours for the next 48 hours. Anti-inflammatory pain killers were avoided.

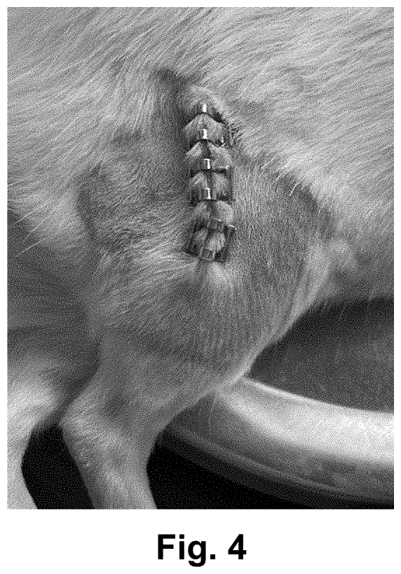

Treatment group 2, 48 hours post-op. As shown in FIG. 4, 48 hours after treatment with the 0.332031% phosphoric acid solution, animals showed a partial reduction in swelling. The site appeared bluish and wound healing margins were not closing-up as would be expected in healthy tissue healing. Fur growth was noticeable, but not to the degree expected in healthy animals. Animals were active and limping was reduced.

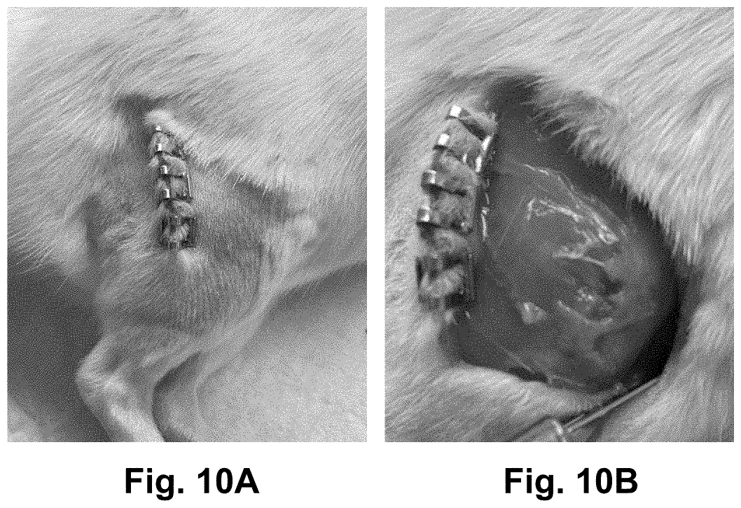

Treatment group 2, 72 hours post-op. FIG. 10A shows the unopened surgical site and FIG. 10B shows the opened surgical site of an animal 72 hours after treatment with the 0.332031% phosphoric acid solution. A reduction in swelling was observed and internal wound tissues showed healing. The tissues adjacent to the wound appeared healthy.

Treatment Group 3

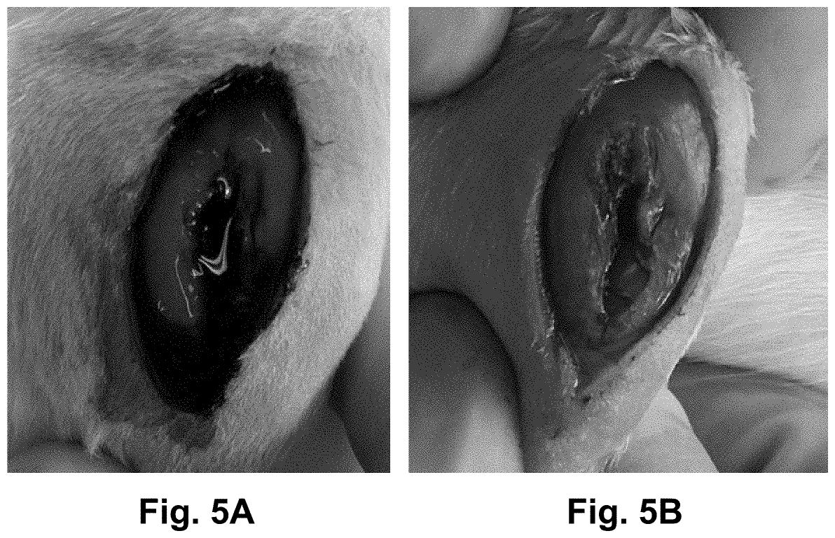

FIG. 5A shows the surgical site after application of the solution containing both 1.328125% phosphoric acid and 0.015625% methylene blue to the entire open surgical site after 5 minutes. Application of the solution containing both 1.328125% phosphoric acid and 0.015625% methylene blue did not stop bleeding entirely (approximately 95% of the bleeding was stopped).

FIG. 5B shows the surgical site after the after the 5 minute application of solution containing both 1.328125% phosphoric acid and 0.015625% methylene blue and after being rinsed with sterile water. No bleeding was observed. The infected tissue area is stained blue and also appears “curled.” Only stained and curled tissues were surgically removed. The site was rinsed again with sterile water until completely stain-free and the wound was closed with internal resorbable stiches and skin staples. Animals received 1 ml saline solution subcutaneously and 0.03 mg/kg of buprenorphine for pain. Buprenorphine was then administered at 0.02 mg/kg every 12 hours for the next 48 hours. Anti-inflammatory pain killers were avoided.

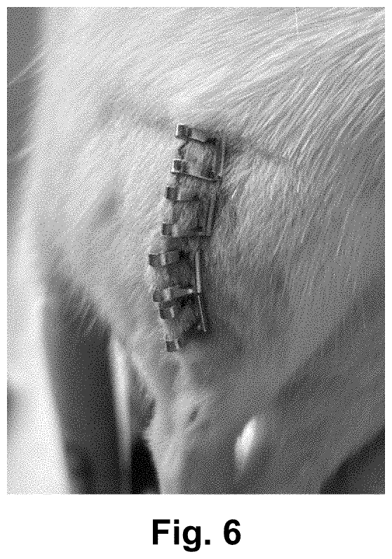

Treatment group 3, 48 hours post-op. As shown in FIG. 6, 48 hours after treatment with the solution containing both 1.328125% phosphoric acid and 0.015625% methylene blue, animals showed a noticeable reduction in swelling and bluish color. Fur growth appeared as would be expected in a healthy animal. Limping was noticeably reduced and animals were lively and rearing, putting full weight on the hind leg that underwent surgery.

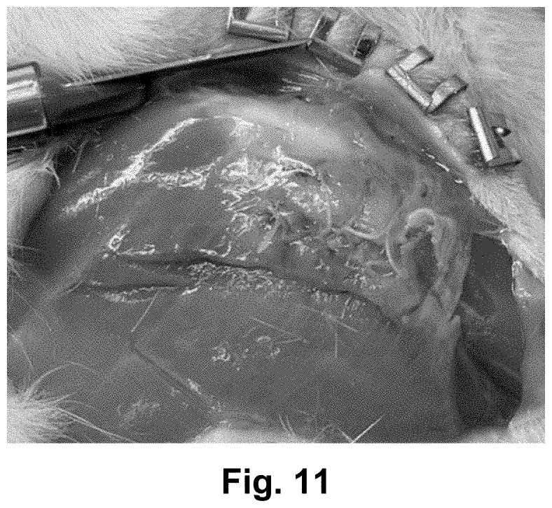

Treatment group 3, 72 hours post-op. FIG. 11 shows the opened surgical site of an animal 72 hours after treatment with the solution containing both 1.328125% phosphoric acid and 0.015625% methylene blue. Apart from an accidental incision that occurred during opening of the surgical site, the wound appeared filled with healthy tissues. All adjacent tissues appeared perfectly healthy.

Treatment Group 4

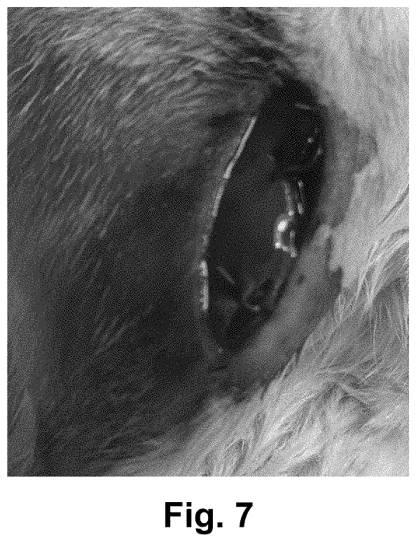

FIG. 7 shows the surgical site after being surgically opened. Since there were no indicators of the extent of infected tissues, a much larger area of the tissues were resected, leaving a noticeable tissue dehiscence. After debridement, the wound was rinsed with sterile water. The wound continued bleeding throughout, even after the application of sutures. Pressure using a sterile cotton gauze was applied to stop the bleeding. The wound was closed with internal resorbable stiches and skin staples. Animals received 1 ml saline solution subcutaneously and 0.03 mg/kg of buprenorphine for pain. Buprenorphine was then administered at 0.02 mg/kg every 12 hours for the next 48 hours. Anti-inflammatory pain killers were avoided.

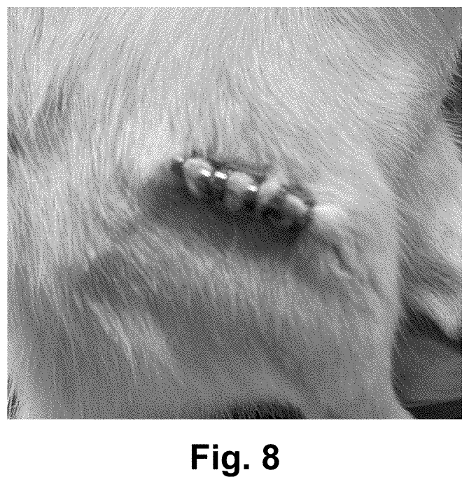

Treatment group 4, 48 hours post-op. As shown in FIG. 8, 48 hours after being treated with conventional debridement, the wound healing appears healthy.

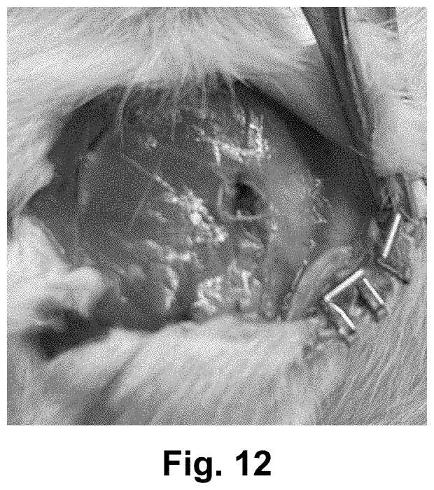

Treatment group 4, 72 hours post-op. FIG. 12 shows the opened surgical site of an animal 72 hours after treatment using conventional debridement. The surgical site was not closing up and was oozing. The surrounding tissues appeared to be dark in color (chronic inflammation).

Treatment Group 5

Treatment group 5, the negative control group, received no treatment after being infected at the first surgery and did not undergo the second surgery. 48 hours after the first surgery, animals received 1 ml saline solution subcutaneously and 0.03 mg/kg of buprenorphine for pain. Buprenorphine was then administered at 0.02 mg/kg every 12 hours for the next 48 hours. Anti-inflammatory pain killers were avoided.

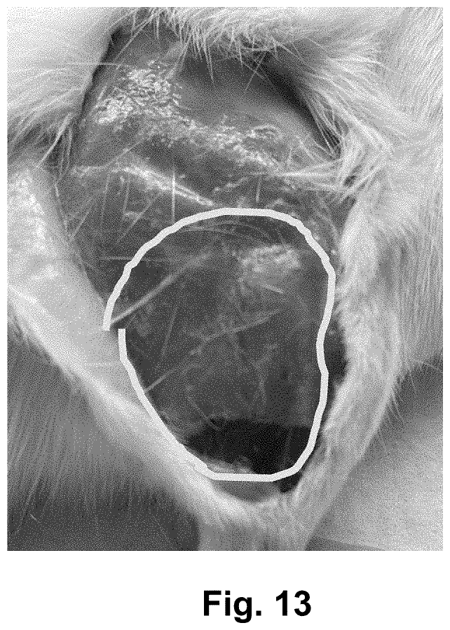

Treatment group 5, 120 hours post-infection. FIG. 13 shows the opened infected site of an animal 120 hours after being infected (the equivalent to 72 hours post-op for the other treatment groups). The infected tissues were swollen and appeared a different color than the healthy tissue, as can be seen in the area circled in yellow in FIG. 13.

External Healing at 72 Hours Post-Op (120 Hours after Infection)

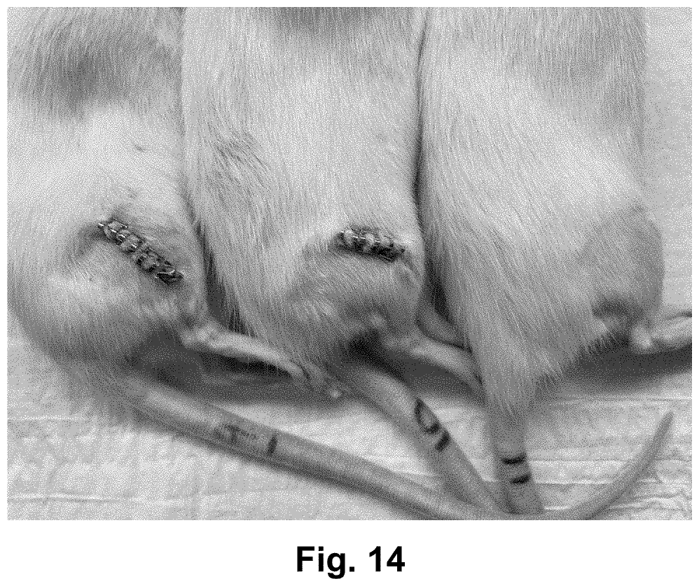

FIG. 14 shows a comparison 72 hours post-op (120 hours after infection for the negative control) of an animal from treatment group 3 (left-most rat), treatment group 4 (center rat), and treatment group 5 (right-most rat). As can be seen, the animal from treatment group 3 (treated with the solution containing both 1.328125% phosphoric acid and 0.015625% methylene blue) showed a considerable reduction of swelling that was practically reduced to the natural, healthy shape of the thigh. The animal from treatment group 4 (treated with conventional debridement) showed swelling of the surgical site. The animal from treatment group 5 (no treatment negative control) had the most swelling of the infected site.

In conclusion, animals of treatment group 3 displayed the fastest healing, anti-inflammatory, and regeneration rate. 72 hours post-op, the wound was closed externally and internally with the healthy tissues. Animals in treatment group 2 showed the second-best healing. Animals in treatment group 1, displayed significant irritation of the tissues, even after being rinsed out thoroughly after the application of the 0.5% methylene blue solution. 72 hours post-op, the treatment group 1 wound was not healing well and showed signs of inflammation. Animals of treatment group 4 showed signs of inflammation and internal healing was delayed.

Interestingly, the low pH of the treatment solutions used for treatment group 2 and treatment group 3 does not appear to affect cells toxically, but to the contrary, appears to facilitate faster regeneration and healing (the mechanism of action is unknown).

### Example 21: In Vivo Studies Using Bioabsorbable Sponges

Healthy Sprague Dawley female rats (Charles River Laboratories, Wilmington, Mass.) 6 months of age (four groups, five per group), were used for study. The rats were infected with the MRSA (Staphylococcus aureus ATCC BAA-1762) at a concentration of 4×10⁸ per ml. The rats were sedated. A small incision was made at a femur site and a slow rotor drill was used to create a dehiscence/trauma in the femoral bone. MRSA bacteria (four microliters) were introduced into the bone and surrounding soft tissues. The wounds were then closed with internal stitches and skin clips. All rats showed severe signs of infection after 72 hours—swelling and cyanotic color of this site, ruffled fur, loss of weight, hunched posture, decreased mobility. An MRI scan showed change in soft tissue at the infected site.

A second surgery/treatment was performed 72 hours post infection. Rats were sedated and outer skin was cleaned with iodine. Skin staples were removed and the wounds reopened. Infected sites showed purulent exudate, granulation of tissue, and excessive bleeding. Most of the granulated tissue and oxidate were removed with a curette. Tissue samples were taken for a histological evaluation.

The initial treatment was performed by using 37% phosphoric acid gel and methylene blue carried by bioabsorbable sponge (GELFOAM®) applied onto the entire area of the wound for five minutes. After five minutes, the sponge was removed. Then wound was rinsed out with saline solution. Wound was not bleeding. The bacteria present were stained black visually demarcating the line of the infected tissues. They were surgically removed. The residual black stained tissues were removed by using the ultrasound machine with the smooth up to avoid tissue damage. The ultrasound waves cleaned the deepest layers of the tissue and restored a fresh blood flow. A bioabsorbable sponge (GELFOAM®, Pfizer, Inc.) containing an aqueous solution of phosphoric acid and methylene blue was inserted into the wound and the wounds were closed.

The treated tissues had stopped bleeding and the infected sites were clearly delineated since methylene blue has an affinity to bind and stain bacteria. Residual infected tissues were surgically removed. An ultrasound machine with saline/water irrigation was used for final cleaning of tissue and to stimulate blood flow.

Rats in Group #1 received bioabsorbable sponge soaked with an aqueous solution containing 0.5 weight percent phosphoric acid and 1 weight percent methylene blue. The wounds were then closed with internal resorbable stitches and skin clips.

Rats in Group #2 received bioabsorbable sponge soaked with an aqueous solution containing 2 weight percent phosphoric acid and 1 weight percent methylene blue. The wounds were then closed with internal resorbable stitches and skin clips.

Rats in Group #3 received a bioabsorbable sponge soaked with an aqueous solution containing 5 weight percent phosphoric acid and 1 weight percent methylene blue. The wounds were then closed with internal resorbable stitches and skin clips.

Rats in Group #4 received a bioabsorbable sponge soaked with a aqueous solution containing 25 weight percent phosphoric acid and 1 weight percent methylene blue. The wounds were then closed with internal resorbable stitches and skin clips.

All four groups of rats showed signs of recovery in the next 24 hours. Rats in Groups #1-#3 showed rapid healing. A healthy change in fur, mobility, reduction of swelling and redness of the wound site were observed. On Day 3 post-op, the rats started to gain weight in a continuous manner, at an average of about 2 to 3 grams per day. On Day 3 post-op rats fur had restored their initial healthy shine and condition, rats did not display any signs of pain or disease, were rearing using both legs. The wound site was healed within 30 days post-infection. The MRL CT scan and cell histology results showed no signs of infection and inflammation on the site. All evaluations showed presence of the new healthy tissue formed, new bone formation and bone density increase. The MRI scan done on the tenth post-operative day showed that infected site volume had decreased by 62% and new tissues were formed.

Rats in Group #4 showed signs of secondary infection starting on Day 3 post-op. These rats began to rip at the skin clips, the wounds were opened, and a purulent oxidate was observed. MRI scan showed a reduction of the MRSA created tissue destruction and the presence of new, superficial tissue damage was observed as well. The rats in Group #4 were euthanized, and pathology revealed soft tissue inflammation around partially disintegrated bioabsorbable sponge.

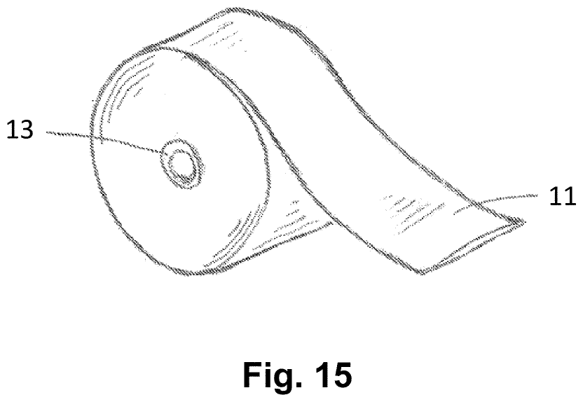

FIG. 15 shows a roll of bioabsorbable sponge 11 on a core 13. For wound treatment, a portion of the bioabsorbable sponge is soaked in an aqueous solution of phosphoric acid and methylene blue, and the like thiazine dye, prior to introduction into a wound.

Various embodiments of the present invention may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:

P1. A therapeutic composition comprising:
1.0×10⁻⁷-2.5×10⁻¹% (w/w) thiazine dye, and
1.0×10⁻⁵-2.0% (w/w) phosphoric acid.

P2. The therapeutic composition of claim P1, wherein the thiazine dye is methylene blue.

P3. The therapeutic composition of any one of claims P1-P2, wherein the therapeutic composition is a mouthwash.

P4. The therapeutic composition of any one of claims P1-P2, wherein the therapeutic composition is a nasal spray.

P5. The therapeutic composition of any one of claims P1-P2, wherein the therapeutic composition is a topical cream.

P6. The therapeutic composition of any one of claims P1-P2, wherein the therapeutic composition is a lozenge.

P7. A method of treating a subject in need thereof, the method comprising:

causing the administration of the therapeutic composition of any one of the preceding claims, wherein the subject is selected from the group consisting of a human and an animal.

P8. A bioabsorbable antimicrobial and anti-inflammatory composition comprising:

a bioabsorbable water-insoluble sponge containing an aqueous solution of phosphoric acid and a thiazine dye, wherein phosphoric acid concentration is in the range of about 0.1 to about 40 percent by weight of the aqueous solution.

P9. The bioabsorbable composition in accordance with claim P8, wherein the phosphoric acid concentration is about 0.1 to about 2.5 percent by weight of the aqueous solution.

P10. The bioabsorbable composition in accordance with claim P8, wherein the phosphoric acid concentration is about 5 to about 40 percent by weight of the aqueous solution.

P11. The bioabsorbable composition in accordance with claim P8, wherein the thiazine dye methylene blue.

P12. The bioabsorbable composition in accordance with claim P10, wherein methylene blue is present in the aqueous phosphoric acid solution in an amount in the range of about 0.0001 percent by weight to about 1 percent by weight, based on the weight of the aqueous phosphoric acid solution.

P13. The biocompatible composition in accordance with claim P10, wherein methylene blue is present in the aqueous phosphoric acid solution in an amount in the range of about 0.1 to about 1 percent by weight of the aqueous phosphoric acid solution.

P14. The bioabsorbable composition in accordance with claim P8, wherein the sponge comprises gelatin.

P15. The bioabsorbable composition in accordance with claim P8, wherein the sponge comprises collagen.

P16. The bioabsorbable composition in accordance with claim P8, wherein the sponge contains about 5 to about 40 percent by weight of the aqueous phosphoric acid solution, based on the weight of the bioabsorbable sponge.

P17. The bioabsorbable sponge in accordance with claim P8, wherein the aqueous solution contains a biocompatible thickening agent.

P18. The bioabsorbable sponge in accordance with claim P17, wherein the thickening agent is sodium carboxymethyl cellulose.

P19. An antiviral textile material comprising:
a hydrophilic fibrous substrate;
an antiviral thiazine dye;
and phosphoric acid;
the hydrophilic fibrous substrate having porosity sufficient to permit a human to breathe therethrough, having said thiazine dye distributed therewithin in an amount in the range of 0.001 to about 1 grams per cubic centimeter of said substrate, and having phosphoric acid distributed therewith in an amount in the range of about 0.001 to about 1 grams per cubic centimeter of said substrate.

P20. The antiviral textile material in accordance with claim P19 wherein the antiviral thiazine dye is methylene blue.

P21. The antiviral textile material in accordance with claim P20 wherein the methylene blue and the phosphoric acid are present in equimolar amounts.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A therapeutic composition comprising:
1.0×10⁻⁷-2.5×10⁻¹% (w/w) thiazine dye, and
1.0×10⁻⁵-2.0% (w/w) phosphoric acid.

2. The therapeutic composition of claim 1, wherein the thiazine dye is methylene blue.

3. The therapeutic composition of claim 1, wherein the therapeutic composition is a mouthwash.

4. The therapeutic composition of claim 1, wherein the therapeutic composition is a nasal spray.

5. The therapeutic composition of claim 1, wherein the therapeutic composition is a topical cream.

6. The therapeutic composition of claim 1, wherein the therapeutic composition is a lozenge.

7. A bioabsorbable antimicrobial and anti-inflammatory composition comprising:
a bioabsorbable water-insoluble sponge containing an aqueous solution of phosphoric acid and a thiazine dye, wherein phosphoric acid concentration is in the range of about 0.1 to about 40 percent by weight of the aqueous solution.

8. The bioabsorbable composition in accordance with claim 7, wherein the phosphoric acid concentration is about 0.1 to about 2.5 percent by weight of the aqueous solution.

9. The bioabsorbable composition in accordance with claim 7, wherein the phosphoric acid concentration is about 5 to about 40 percent by weight of the aqueous solution.

10. The bioabsorbable composition in accordance with claim 7, wherein the thiazine dye methylene blue.

11. The bioabsorbable composition in accordance with claim 9, wherein methylene blue is present in the aqueous phosphoric acid solution in an amount in the range of about 0.0001 percent by weight to about 1 percent by weight, based on the weight of the aqueous phosphoric acid solution.

12. The biocompatible composition in accordance with claim 9, wherein methylene blue is present in the aqueous phosphoric acid solution in an amount in the range of about 0.1 to about 1 percent by weight of the aqueous phosphoric acid solution.

13. The bioabsorbable composition in accordance with claim 7, wherein the sponge comprises gelatin.

14. The bioabsorbable composition in accordance with claim 7, wherein the sponge comprises collagen.

15. The bioabsorbable composition in accordance with claim 7, wherein the sponge contains about 5 to about 40 percent by weight of the aqueous phosphoric acid solution, based on the weight of the bioabsorbable sponge.

16. The bioabsorbable sponge in accordance with claim 7, wherein the aqueous solution contains a biocompatible thickening agent.

17. The bioabsorbable sponge in accordance with claim 16, wherein the thickening agent is sodium carboxymethyl cellulose.

18. An antiviral textile material comprising:
a hydrophilic fibrous substrate;
an antiviral thiazine dye;
and phosphoric acid;
the hydrophilic fibrous substrate having porosity sufficient to permit a human to breathe therethrough, having said thiazine dye distributed therewithin in an amount in the range of 0.001 to about 1 grams per cubic centimeter of said substrate, and having phosphoric acid distributed therewith in an amount in the range of about 0.001 to about 1 grams per cubic centimeter of said substrate.

19. The antiviral textile material in accordance with claim 18 wherein the antiviral thiazine dye is methylene blue.

20. The antiviral textile material in accordance with claim 19 wherein the methylene blue and the phosphoric acid are present in equimolar amounts.
